SEQUENCE LISTING (13 sequences)

SEQ ID NO: 1 - DNA, 37 bp, Artificial Sequence
  primer
cccgggccatggctagcaaaggagaagaacttttcac

SEQ ID NO: 2 - DNA, 36 bp, Artificial Sequence
  primer
ggccgcggccgcttatttgtagagctcatccatgcc

SEQ ID NO: 3 - DNA, 51 bp, Artificial Sequence
  primer
ctagcgtgagcgctcacaggtcaattcggtgagcgctcacaggtcaattcg

SEQ ID NO: 4 - DNA, 52 bp, Artificial Sequence
  primer
ctagcggaccaggacaaaggtcacgttcggaccaggacaaaggtcacgttcg

SEQ ID NO: 5 - DNA, 70 bp, Artificial Sequence
  primer
cccgggtaactataacggtcctaaggtagcgagcaattgttgttaacttgtttattgcagcttataatgg

SEQ ID NO: 6 - DNA, 36 bp, Artificial Sequence; identical to SEQ ID NO: 2
  primer

SEQ ID NO: 7 - DNA, 36 bp, Artificial Sequence
  primer
atcggcggcgccgccatgaaacaacttgaagacaag

SEQ ID NO: 8 - DNA, 37 bp, Artificial Sequence
  primer
gatcaaagcttgcggccgctcagcgttcgccaactaa

SEQ ID NO: 9 - DNA, 59 bp, Artificial Sequence
  primer
gaagaagggatcctggtgcctcgtggttctgccatgggcactacaaatactgtggcagc

SEQ ID NO: 10 - DNA, 57 bp, Artificial Sequence
  primer
gaagaagggatcctggtgcctcgtggttctgccatggagacccctgcctggccccgg

SEQ ID NO: 11 - DNA, 29 bp, Artificial Sequence
  primer
ggcggcgccgcctatttctcgaattcccc

SEQ ID NO: 12 - DNA, 46 bp, Artificial Sequence
  primer
ggcggcgccgcctatttctcgcccatacactctaccgggctgtctg

SEQ ID NO: 13 - DNA, 48 bp, Artificial Sequence
  primer
ggcggcgccgcctatttctcctctaccgggctgtctgtactcttccgg

(19) United States
(12) Patent Application Publication
(10) Pub. No.: US 2010/0099108 A1
(43) Pub. Date: Apr. 22, 2010

(54) Method for Detecting, Isolating, and Characterizing Cells from Body Samples by Transfection with Nucleic Acid Constructs

(75) Inventor: Ralph Wirtz, Koln (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/578,618

(22) Filed: Oct. 14, 2009

Related U.S. Application Data
(63) Continuation of application No. 10/474,078, filed on Mar. 19, 2004, now abandoned.

(30) Foreign Application Priority Data
Apr. 2, 2001 (DE) 10116323.1
Jul. 12, 2001 (DE) 10133930.5
Dec. 7, 2001 (DE) 10160271.5

(51) Int. Cl. C12Q 1/68

(52) U.S. Cl. 435/6

(57) ABSTRACT

A method for detecting or isolating disease-associated cells or pluripotent stem cells from body samples is provided where cells of a body sample are transfected with nucleic acid constructs that include the following components: (a) a promoter element containing at least one DNA site for binding one or more transcription factors; and (b) a reporter gene that enables the diseased or stem cells to be detected. When the method is used for detecting disease-associated cells, the transcription factor initiates at least one signalling activity in the disease-associated cells that is not present in healthy cells.

### FIELD OF THE INVENTION

The present invention relates generally to a method for detecting specific cells in a body sample and where appropriate, for isolating the cells from a living organism and making such cells available for clinical investigations or therapeutic applications. Within the context of the present invention, the specific cells may be disease-associated cells such as tumor cells and the body sample may be a tissue or blood sample.

### BACKGROUND OF THE INVENTION

After cardiac and circulatory diseases, cancer and malignant neoplasias are, by a wide margin, the second most frequent cause of death in Germany and other industrialized nations of the world. The majority of all new cases of cancer and of malignancy-associated death in western industrialized countries are caused by malignant epithelial tumors. In the European Union, approximately 577,000 people fall ill with cancer every year and approximately 376,000 people die every year from the most frequent solid tumor types (breast carcinoma, prostate carcinoma, lung carcinoma and colon carcinoma). If the breakneck decline in mortality associated with cardiac and circulatory diseases is to continue, it can then be expected that cancer will become the most frequent cause of death in Germany within about 15-20 years.

Because of improvements in tumor excision over the last few decades, the mortality rate from cancer is increasingly being determined by metastasis behavior and premature occult tumor cell dissemination. Because metastasis behavior and premature occult tumor cell dissemination are both difficult or impossible to detect due to the insensitive nature of conventional histopathological staging methods, there is a need for the detection of tumor cells in blood. The detection of metastatic cells in blood or lymph nodes may indicate the malignancy of a cancerous change and precise characterizations of tumor cells in blood may aid decision-making with respect to appropriate therapies.

Due to specificity problems associated with conventional detection methodologies (i.e., immunocytochemistry, in situ hybridization, or PCR), it is extremely difficult to identify, characterize, and isolate tumor cells by detecting genes that are expressed in a tumor-specific manner. This is due, inter alia, to the fact that the expression of specific tumor markers is frequently restricted to particular tumor types or the heterogeneous cell populations in a tumor are such that it is only possible to recognize some of the tumor cells. The detection of rare events in the blood such as metastatic cells also comes up against technical difficulties, such as low sensitivity; problems with antibody specificity; or increasing costs. In this connection, efficient detection of abnormal cells, or the separation of living tumor cells from healthy cells in a mixture of the two cell types, would be of great value for therapeutic as well as diagnostic purposes. Thus, it is important to be able to determine, as precisely as possible, at what time chemotherapies, for example, should be commenced or terminated. It is also of great interest to ascertain the efficacy of medicaments during the course of a therapy. For this, it is necessary to have available a method, which is as sensitive and rapid as possible, for qualitatively detecting the presence of even very small quantities of metastatic cells.

The methods developed by the companies Immunicon and Miltenyi Biotec are those which are the furthest advanced in this area; these companies use antibodies directed against epithelial surface antigens (EpCAM and/or HEA) in magnetic cell separation methods. The antigens are expressed to differing degrees on the cell surfaces of all epithelial cells, including both healthy cells and malignant cells. Since blood cells do not possess these antigens, these methods enable epithelial cells to be concentrated from approximately 5 to 10 times from the blood. Due to the small number of malignant cells in the tumor sample, the majority of the cells still consist of peripheral blood cells following magnetic separation. Thus, Krüger et al. report using the HEA immunobead methodology to concentrate epithelial cells from 2.6 per 10⁶ cells to 10.6 per 10⁶ cells, with a caveat being that there is a significant loss of tumor cells. Krüger et al., CYTOTHERAPY 1: 135-139 (1999). Moreover, this methodology cannot be used to concentrate tumor types which are not of epithelial origin. Other methods for identifying carcinoma cells in blood samples include steps for permeabilizing and fixing cells, which means that it is not possible to isolate any living tumor cells. In this connection, the positively selected cells are stained using immunocytochemical methods, with antibodies directed against cytokeratins for the most part being employed for detecting epithelial cells. Since the corresponding cytokeratins are not present in blood cells, the detection of this protein, which is present in all epithelial cells, counts as the detection of metastatic tumor cells. These methods do not only come up against technical difficulties, they are also costly, personnel-intensive and time-consuming.

The replication of body cells is normally controlled by a large number of regulatory mechanisms within the cell such that each cell only begins to divide when required and in harmony with the organism as a whole. These processes are regulated by signal molecules which are secreted by “transmitter cells,” which bind to specific receptor molecules on the surface of the “receptor cells,” and which subsequently activate what are termed signal transduction pathways that ultimately result in altered gene expression. In tumor cells, activating or inhibitory regulatory molecules that control these signalling activities (the oncogenes or tumor suppressor genes correspond to them) are frequently mutated. Consequently, tumor cells possess activities in their cell nuclei that are otherwise only found in particular developmental stages in embryonic development or in precisely defined tissue regions.

Examples of such signalling activities, which are associated with cancerous growth, are the Wnt and Ras signal transduction pathway and the functional loss of the tumor suppressor p53. In cancerous events in human colon tumors, these signalling activities play a role in different stages in the development of the tumor such that they are diagnostically suitable for staging the tumor cells or for simultaneously analysing several cancer-associated signalling activities. See, e.g., K. W. Kinzler & B. Vogelstein, Lessons from Hereditary Cancer, CELL 87:159-170 (1996). Components of the Wnt signal transduction pathway are already mutated in virtually all the colon carcinoma cells at a very early stage. For example, the tumor suppressor gene adenomatous polyposis coli (“APC”) is inactivated by mutations in 80% of spontaneous tumors and the β-catenin oncogene is activated by mutation in 10% of tumors. In all probability, the remaining 10% of the tumors possess mutations in other components of the Wnt signal transduction pathway such that it can in general be assumed that these mutations occur as an early event in the initiation of tumorigenesis. Consequently, Wnt signalling activity, which is ultimately to be ascribed to the reciprocal action of the β-catenin oncogene on the transcription factors in the cell nucleus known as T cell factor (“TCF”) and lymphoid enhancing factor (“LEF”), is present in all colon carcinoma cells. In more advanced stages of colon carcinogenesis, the oncogene K-ras is also activated by mutation in 70% of cases. Normally, the Ras proteins are stimulated after the binding of specific ligands to different cell surface molecules (e.g., receptor tyrosine kinases, integrins, and ion channels) by way of adapter proteins (e.g., Shc, Grb2, Crk, etc.) and downstream guanine nucleotide exchange factors (e.g., Sos, C3G, etc.). Due to mutations of the Ras gene in colon tumor cells, active Ras proteins are expressed constitutively, with these Ras proteins even being active without the upstream components of the Ras signal transduction pathway. This ultimately results in the activation of downstream components of the signal pathway, with this activation resulting in the hyperactivity of a large number of transcription factors (CREB, SRF, cFos, c-Jun, PPAR, ER, ETS, ELK-1, STAT, Myc, Max, DPC4, p53, NFAT4, CHOP, MEF2, ATF-2, etc.) in the cell nucleus, which factors are thereupon able, inter alia, to induce dividing growth of the cells. In an even more advanced colon tumor stage, the tumor suppressor gene p53 is mutated in more than 80% of the tumors. In healthy cells and as an active transcription factor, the p53 gene either prevents the dividing growth of cells when disease-associated changes occur or leads to the programmed death of the cell by binding to specific DNA sequences (PuPuC(A/T)(A/T)GpyPyPy) and activating growth-inhibiting genes (e.g., p21^{CIP1}).

In addition to directly activating transcription factors, tumor-associated signalling activities also frequently lead to the increased expression of transcription factors, which are able to activate or suppress other genes and are consequently only activated secondarily or indirectly. An outstanding example of this is the peroxisome proliferator activated receptor delta (“PPARδ”) transcription factor, which is expressed in colon carcinoma cells as a result of hyperactivity of the Win signal transduction cascade. It has been demonstrated that inhibitors of this transcription factor, i.e., non-steroidal anti-inflammatory drugs (“NSAIDS”) such as sulindac or aspirin, are able to inhibit the growth of tumor cells by inhibiting this transcription factor. T. C. He, T. A. Chan, B. Vogelstein & K. W. Kinzler, PPARdelta is an APC-Regulated Target of Nonsteroidal Anti-Inflammatory Drugs, CELL 99:335-345 (1999). Another example of a secondarily induced transcription factor is c-myc, which is also overexpressed in cells exhibiting active Wnt signalling activity. T. C. He et al., Identification of c-myc as a Target of the APC Pathway, SCIENCE 281:1509-1512 (1998). The synergism of different signalling activities in the progression of tumors was described by Sinn et al. in 1987 using the example of the oncogenes myc and ras. E. Sinn, W. Muller, P. Pattengale, I. Tepler, R. Wallace & P. Leder, Coexpression of MMTV/v-Ha-Ras and MMTV/c-Myc Genes in Transgenic Mice Synergistic Action of Oncogenes in vivo, CELL 49:465-475 (1987). The interaction of protooncogenes (such as β-catenin, Ras, Myc, and Fos) and tumor suppressor genes (such as APC, Rb, and p53) is graphically described in the review article by Hanahan et al. D. Hanahan & R. A. Weinberg, The Hallmarks of Cancer, CELL 100:57-70 (2000).

The signalling activities described above play an important role in a large number of different tumor types. Thus, the number of findings indicating that components of the Wnt signal transduction cascade are mutated in many tumors or that nucleus-located β-catenin is immunohistochemically detectable in tumor tissues has grown exponentially in recent years. This underscores the central and general importance of these signalling activities in cancerous events in human tumors. Thus, activating mutations of the β-catenin oncogene are found, inter alia, in tumors of the liver, the kidney, the pancreas, the stomach, the prostate, the thyroid gland, the uterus, the skin, and in medulloblastomas. For example, β-catenin is mutated in 75% of human skin tumors and in 89% of human liver tumors. E. F. Chan, U. Gat, J. M. McNiff & E. Fuchs, A Common Human Skin Tumour is Caused by Activating Mutations in β-catenin, NAT. GENET. 21:410-413 (1999); Y. M. Jeng, M. Z. Wu, T. L. Mao, M. H. Chang, & H. C. Hsu, Somatic Mutations of β-catenin Play a Crucial Role in the Tumorigenesis of Sporadic Hepatoblastoma. CANCER LETT. 152:45-51 (2000). As previously discussed, mutations are also found in other components of the Wnt signal transduction cascade that have an identical effect with regard to the development of cancer. See, S. Satoh, Y. Daigo, Y. Furukawa, T. Kato, N. Miwa, T. Nishiwaki, T. Kawasoe, H. Ishiguro, M. Fujita, T. Tokino, Y. Sasaki, S. Imaoka, M. Murata, T. Shimano, Y. Yamaoka & Y. Nakamura, AXIN1 Mutations in Hepatocellular Carcinomas and Growth Suppression in Cancer Cells by Virus-Mediated Transfer of AXIN1, NAT. GENET. 24:245-250 (2000). Activation of the Wnt signal transduction cascade also plays a central role in breast tumors and is of prognostic significance. S. Y. Lin, W. Xia, J. C. Wang, K. Y. Kwong, B. Spohn, Y. Wen, R. G. Pestell & M. C. Hung, Beta-Catenin, a Novel Prognostic Marker for Breast Cancer: Its Roles in Cyclin D1 Expression and Cancer Progression, PROC. NAT. ACAD. SCI. USA 97:4262-4266 (2000).

The central importance of p53 in the development of a large number of tumors has been described. The p53 gene is the tumor suppressor gene that is most frequently mutated in human tumors (more than 10,000 mutations have been described in the literature). T. Hernandez-Boussard, P. Rodriguez-Tome, R. Montesano & P. Hainaut, IARC p53 Mutation Database: A Relational Database to Compile and Analyse p53 Mutations in Human Tumours and Cell Lines, HUM. MUTAT. 14:1-8 (1999). The p53 gene regulates cell cycle control and apoptosis in connection with repair mechanisms following DNA damage. Consequently, p53 is also termed the guardian of the genome, with the ability of p53 to function as a transcription factor being of crucial importance on this matter. Mutations of p53 have been found in many tumor types, for example, they have been found in tumors of the colon, the liver, the breast, the stomach, the pancreas, the blood, the lung, and the thyroid gland. The general tumor-suppressor function of p53 is demonstrated in patients with inherited mutations of the p53 gene and who develop a large number of different tumors. S. Mazoyer, P. Lalle, C. Moyret-Lalle, C. Marcais, S. Schraub, D. Frappaz, H. Sobol, & M. Ozturk. Two Germ-Line Mutations Affecting the Same Nucleotide at Codon 257 of p53 Gene, A Rare Site for Mutations, ONCOGENE 9: 1237-1239 (1994); Akashi, M. & Koeffler, H. P., Li-Fraumeni Syndrome and the Role of the p53 Tumour Suppressor Gene in Cancer Susceptibility, CLIN. OBSTET. GYNECOL. 41:172-99 (1998). In addition, mutations in p53 are also responsible for, inter alia, resistances to chemotherapeutic agents. T. Aas, A.-L. Borresen, S. Geisler, B. Smith-Sorenson, H. Johnsen, J. E. Varhaug, L. A. Akslen & P. E. Lonning, Specific P53 Mutations are Associated with de novo Resistance to Doxorubicin in Breast Cancer Patients, NATURE MED. 2: 811-814 (1996).

The central importance of the Ras signal transduction cascade, which is also termed the SOS-Ras-Raf-MAPK cascade, has been described on many occasions in the literature. It has been possible to detect structurally altered Ras proteins, which are tantamount to an incessant growth-promoting signal, in approximately 25% of human tumors. Hanahan et al., The Hallmarks of Cancer, supra. In particular, as previously noted, in colon tumors, the frequency of mutation is very high in particular cancer stages; however, mutations of Ras genes have also been detected in a large number of other tissues, for example, they have been found in tumors of the lung, stomach, pancreas, gall bladder, breast, uterus, and in sarcomas.

The biochemical processes of established cell lines has been studied through the use of reporter gene constructs. Specifically, the reporter gene constructs have been used to detect the signalling activities of activated or secondarily induced transcription factors. Such reporter gene constructs consist of a promoter region, to which particular transcription factors are able to bind, and a reporter gene, which is not normally present in the cells and which when expressed can be easily detected on the basis of either enzymatic activity or fluorescence of the gene product. In the 2001 Clontech catalogue (pp. 210 to 212), Clontech offers for sale a Mercury™ Pathway Profiling System, which can be used to detect the activities of the transcription factors NFAT, AP1, NFκB, CREB, ATF, c-Jun, c-Fos and ELK. According to Clontech, this system is to be used for investigating and quantifying signalling activities in established tumor cell lines. In this connection, the signalling activities are induced by adding external stimuli, such as PMA, ionomycin, and forskolin, and are measured with the aid of the reporter genes luciferase, the secreted form of alkaline phosphatase (“SEAP”), or the destabilized form of enhanced green fluorescent protein (“d2EGFP”). In these assays, the calcium phosphate precipitation methodology is used to transfect the reporter gene constructs transiently into the tumor cell lines in order to investigate the activity of gene constructs that have additionally been transfected into the cells and whose gene products might be able to interact with the signalling activities. Consequently, these investigative approaches have the aim of clarifying the molecular mechanism of the signal transduction cascades.

The use of these reporter gene systems in clinical diagnosis has not been possible because transfection efficiencies (i.e., DNA-mediated gene transfer) of primary tumor cells are not adequate. Further, the promoter elements of primary tumor cells are not optimized to enable the reporter genes to be adequately expressed as a sensitive measurement system, with or without external stimuli. In this same way, while experimental approaches always compare activities of induced and uninduced tumor cell lines, they fail to compare the mechanisms of healthy cells versus tumor cells. Accordingly, the aim of reporter gene systems is quite evidently not the clinical diagnosis of tumor cells but, on the contrary, the elucidation of general signal transduction mechanisms in tumor cell line systems.

The fundamental defect in current reporter gene systems is evident in U.S. Pat. No. 5,851,775 to Barker et al., which describes a method for identifying potential therapeutic agents using TCF transcription factor-responsive reporter gene constructs. The Barker et al. research group has previously used luciferase reporter system to detect tumor-associated Wnt signalling activity in colon carcinoma cell lines in cell culture systems. V. Korinek, et al., Constitutive Transcriptional Activation by a Beta-Catenin-TCF Complex in APC−/− Colon Carcinoma, SCIENCE 275:1784-1787 (1997). In the Barker et al. patent, cells which exhibit mutations in the signal transduction components APC or β-catenin were investigated; however, the Barker et al. system was unable to find substances that exert an effect on the Wnt signal cascade as a result of interactions with other components of the cascade. Further, the reporter gene constructs disclosed in the Barker et al. patent were not disclosed for the purpose of detecting or isolating primary tumor cells.

In U.S. Pat. No. 6,140,052, He et al. claims a reporter gene construct for determining Wnt signalling activity. The construct contains a precisely specified base sequence (CTTTGAT and ATCAAAG), derived from the c-myc target gene promoter, for the binding site of TCF transcription factors. This disclosure appears to contradict the fact that TCF transcription factors are insensitive to changes in the base sequence of their DNA binding sites. In 1995, using cocrystallization experiments, Love et al. described the strong DNA-binding activity of the closely related LEF-1 transcription factor with the base sequence (CCTTTGAA) of the DNA binding site. J. J. Love et al., Structural Basis for DNA Bending by the Architectural Transcription Factor LEF-1, NATURE 376: 791-795 (1995). Love et al. reported that the position of the middle thymidine is critical for the binding affinity whereas the transversion (T>A) of the adjacent thymidine has no influence on the binding activity. Thus, it follows that other base sequences would also be similarly efficient in their binding affinities. The LEF-1 and TCF factors are closely related both structurally and functionally and can in fact replace each other functionally. The importance of the LEF-1 and TCF factors in colon carcinogenesis was disclosed in October 1996. K. W. Kinzler & B. Vogelstein, Lessons from Hereditary Cancer, supra.

The green fluorescent protein (“GFP”), which fluoresces when expressed, is suitable for use as a reporter gene for isolating living tumor cells from blood samples. In U.S. Pat. No. 5,968,738 to Anderson et al. signal transduction activities are measured using two GFP variants and flow cytometry (“FACS”). Other patents relating to GFP all deal with practical applications in therapy or cell sorting of cell line mixtures but none deal with GFP as a reporter gene for diagnosing metastatic cells obtained from blood. For example, in 1996, Crameri et al. disclosed a GFP variant (αGFPT204I) with improved fluorescent behaviour; however, this GFP variant was not disclosed as having use as a reporter gene. A. Crameri, E. A. Whitehorn, E. Tate & W. P. C. Stemmer, Improved Green Fluorescent Protein by Molecular Evolution Using DNA Shuffling, NATURE BIOTECH. 14:315-319 (1996). In Crameri et al., a constitutively expressed, red-fluorescing protein derived from Discosoma sp. (“DsRed”) was used as a transfection control; DsRed is supplied by Clontech (Cat. No. 6921-1).

In addition to fluorescent reporter gene products, genes that may be detected on the basis of structural properties may also be used for isolating living tumor cells from body samples. Of particular interest in this connection are genes that encode proteins that may be detected extracellularly. For example, transmembrane proteins that expose antigenic structures that are recognized by corresponding antibodies on cell surfaces are particularly suitable in this connection. Antigenic sequences of nonhuman origin are particularly advantageous in this context since the antibodies will have no crossreactivities and there will be no detectable endogenous human gene expression. Thus, molecular biology methods may be used to recombine the antigenic structures with natural gene sequences such that the antigenic structures are preferably presented extracellularly.

Also suitable for detecting disease-associated signalling activities are genes that mediate enzymatic activities, such as luciferases, β-galactosidases, proteases, glycosidases, acetylases and phosphatases, all of which may be present either intracellularly or secreted. Amplification systems are useful to increase the sensitivity of the methods when they are used to detect rare cellular events. Such application systems are widespread in immunohistochemistry and immunocytochemistry and generally involve the use of secondary antibodies or the biotin-streptavidin system in combination with enzymatic detection reactions to increase the intensity of the signal.

Normally, when injuries occur, the body protects itself against blood loss by the process of hemostasis. Under these circumstances, a cascade of enzymatic reactions is set in motion in the presence of different activating substances. Of particular importance on this matter is the extrinsic activation of the coagulation system by factor VII using tissue thromboplastin as a protein cofactor. In contrast to all the other inactive coagulation factor precursors, the factor VII zymogen, which is circulating in the blood, already possesses proteolytic activity that does not lead to activation of coagulation if no tissue thromboplastin is present. When tissue is damaged, tissue thromboplastin is released from the microsomes of damaged cells. In its natural form, tissue thromboplastin is a complex consisting of a protein and a phospholipid. Without proteolytic activity, tissue thromboplastin that has been released imparts a greater activity to the single-chain factor II zymogen. In the presence of calcium ions, the tissue thromboplastin-factor VII complex forms a complex on phospholipid particles (platelet factor 3), on which complex activation of factors IX and X takes place. Within the course of the progressive activation of the plasma coagulation system, an enormous amplification of the starting signal is associated with the activation of each subsequent enzyme reaction. This avalanche-like reaction cascade, which is also termed plasma coagulation, is terminated by the conversion of fibrinogen to fibrin, whose three-dimensional network permeates the incompact platelet thrombus at the site of the injury and consolidates the thrombus. What is crucial in association with the extrinsic activation of the coagulation cascade is the binding of moieties of the extracellular domain of the tissue thromboplastin to the factor VII zymogen. Naturally occurring tissue thromboplastin is an integral membrane protein which, in intact cells, is concealed in the interior of the cell (in the endoplasmic reticulum) and is only released when the integrity of the cell is violated, which means that it is not possible for the blood coagulation cascade to be activated prematurely. Determination of the clinicochemical parameter thromboplastin time (“Quick value,” “TPT,” or “prothrombin time”) is of clinical relevance. The thromboplastin time is one of the coagulation analyses that has to be carried out before all surgical interventions in order to identify any possible decrease in factors II, V, VII, and X, and in fibrinogen. For this, fibrinogen formation is induced in plasma from citrate whole blood by adding tissue thromboplastin and calcium ions; the coagulation time is determined in comparison with control blood plasma and the thromboplastin time is decreased during administration of heparin.

Because of the molecular constitution of natural tissue thromboplastin, it is a technically elaborate matter to isolate and purify a functional protein/phospholipid particle for the purpose of determining the thromboplastin time. To overcome this difficulty, recombinant water-soluble non-naturally occurring variants of thromboplastin have been prepared in bacteria and other expression systems. See, e.g., J. H. Morrissey, Tissue Factor Modulation of Factor VIIa Activity: Use in Measuring Trace Levels of Factor VIIa in Plasma, THROMB. HAEMOST. 74:185-188 (1995). These water-soluble forms of tissue thromboplastin (“soluble tissue factor” or “sTF”), consisting of amino acids 1 to 219, are commercially available (e.g., as a constituent of a kit supplied by Diagnostics Stago (Cat. No. 00281)) for determining the quantity of factor VIIa and can activate factor X in the presence of phospholipid vesicles and factor VIIa. W. Ruf, A. Rehemtulla, J. H. Morrissey & T. S. Edgington. Phospholipid-Independent and Dependent Interactions Required for Tissue Factor Receptor and Cofactor Function, J. BIOL. CHEM. 266:16256 (1991). In experiments by Neuenschwander et al., the isolated extracellular domain of thromboplastin was expressed in mammalian cells and isolated from cell culture supernatants as a secreted protein. It was found that the loss of the transmembrane region is of importance for the autoactivation of factor VII, resulting in a greatly extended duration of coagulation in standard blood coagulation assays. P. F. Neuenschwander & J. H. Morrissey, J. H. Deletion of the Membrane Anchoring Region of Tissue Factor Abolishes Autoactivation of Factor VII But Not Cofactor Function. Analysis of a Mutant with a Selective Deficiency in Activity, J. BIOL. CHEM. 267:14477-14482 (1992). By fusing a synthetic leucine zipper dimerization domain, a recombinant form of the isolated extracellular domain of thromboplastin has been generated. This recombinant thromboplastic extracellular domain is able to autoactivate factor VII, even in the absence of phospholipids, and also to activate factor X in a manner similar to purified wild-type thromboplastin. F. Donate, C. R. Kelly, W. Ruf, & T. S. Edgington, Dimerization of Tissue Factor Supports Solution-Phase Autoactivation of Factor VII Without Influencing Proteolytic Activation of Factor X, BIOCHEMISTRY 39:11467-11476 (2000).

U.S. Pat. No. 6,080,575 to Heidtmann et al. describes the use of nucleic acid constructs to alter blood coagulation times. The nucleic acid constructs encode a fusion product consisting of an active component, a protease-sensitive region, and an inhibitory component. The active component of the fusion protein can be, inter alia, a component of the blood coagulation cascade such as factor X, which is rendered inactive by the inhibitory component and which is rendered active by the presence of particular proteases in the surrounding medium. Thus, for example, by adding prostate specific antigen (“PSA”) to cell culture supernatants from cells that have been stably transfected with the nucleic acid construct, the active component is released leading to shorter blood coagulation times in blood coagulation assays following recalcification. U.S. Pat. No. 4,784,950 to Hagen et al. discloses the expression of proteins that activate blood coagulation in mammalian cells. The method described therein produces large and highly pure quantities of factor VIIa and factor IX that are used in the treatment of patients that display such deficiencies. While Heidtmann et al. and Hagen et al. use recombinant techniques to adjust blood coagulation times, neither patents contemplates the use of the recombinant techniques to discriminate between healthy and diseased cells.

The isolation of particular cells from heterogeneous cell mixtures is of interest for detecting and characterizing pathologically altered cells and also for diagnostic and therapeutic purposes. For example, the isolation of adult stem cells, which can differentiate into different organ-specific or tissue-specific cells, is of great medical interest. Theoretically, after isolation and replication of stem cells from one organ ex vivo, stem cells could theoretically be used for culturing and autologously transplanting replacement organs after appropriate trandifferentiation. The use of adult stem cells from other organs, however, is extremely complex since it is very difficult to isolate the rare stem cells in an undifferentiated state and at high purity.

Pluripotent cells have been detected in a large number of organs. Thus, it is possible to enrich stem cells from rapidly regenerated organs (such as skin, intestine, and skeletal muscle) under selective growth conditions. According to recent investigations, adult stem cells require specific biochemical activities for maintaining their dedifferentiated state. This is seen in particular in stem cells derived from the epithelium of the small intestine where the stem cells are lost when the transcription factor TCF4 is deleted. V. Korinek, N. Barker, P. Moerer, E. van Donselaar, G. Huls, P. J. Peters & H. Clevers, Depletion of Epithelial Stem-Cell Compartments in the Small Intestine of Mice Lacking Tcf-4, NAT. GENET. 19:379-383 (1998). According to the Korinek et al. study, the stem cells of the small intestine require Wnt signalling activity if they are to continue to exist in the adult body. Wnt signalling activity is mediated by TCF4 in the crypts of the small intestine microvilli. Similar responses may be attributed to other organ systems from investigations in which components of the Wnt signal cascade have been specifically deleted or have been expressed in active form. The Wnt signal cascade also appears to be of importance in cells that are precursors of the hematopoietic system in that Wnt factors appear to regulate the expansion and maintenance of hematopoietic precursor cells. T. W. Austin, G. P. Solar, F. C. Ziegler, L. Liem & W. Matthews, A Role for the Wnt Gene Family in Hematopoiesis: Expansion of Multilineage Progenitor Cells, BLOOD 89:3624-3635 (1997). It has also been demonstrated that adult bone marrow tissue contains stem cells that possess a high degree of plasticity and/or a broad potential for differentiation. D. S. Krause, N. D. Theise, M. I. Collector, O. Henegariu, S. Hwang, R. Gardner, S. Neutzel & S. J. Sharkis, Multi-Organ, Multi-Lineage Engraftment by a Single Bone Marrow-Derived Stem Cell. CELL 105:369-377 (2001). Following transplantation into the recipient organism, specific bone marrow stem cells assume the function of stem cells in the lung, the skin, the liver and the digestive tract. In order to enrich the corresponding stem cells, the authors made use of what is termed a “homing assay,” which involves serially transplanting purified and labelled bone marrow cells into X-ray-irradiated recipient animals. This type of isolation of adult stem cells, however, is not suitable for clinical applications in humans.

According to recent investigations, a large number of tumors appear to develop from adult and/or somatic stem cells. This finding stems ensues from the observation that specific signal cascades (such as Wnt and hedgehog) are required in particular tissues for maintaining cell populations and that aberrant activation of the same signal cascades contributes to the development of a high percentage of particular tumors in the same tissues. J. Taipale & P. A. Beachy, The Hedgehog and Wnt Signalling Pathways in Cancer, NATURE 411:349-354 (2001). The fact that between four and seven mutations have to take place in a single somatic cell for a tumor to be formed suggests that the resulting tumor cells must have been present for a relatively long period of time even in tissues that renew themselves rapidly (such as intestine, skin, and blood). Signalling activities that are causatively involved in tumor development are consequently potentially also active in stem cells. Methods for isolating tumor cells can also be used for isolating specific stem cells.

At the 2001 Wnt Meeting in New York, Tannishtha Reya demonstrated that retroviral expression of β-catenin preserves hematopoietic stem cells in an undifferentiated state over a long period of time and increases their ability to colonize the bone marrow of lethally irradiated mice for the purpose of constructing a complete hematopoietic system. This demonstration clarifies the function and importance of the Wnt signal cascade in the maintenance of hematopoietic stem cells and suggests that the isolation of blood cells from the bone marrow can be used, on the basis of an active Wnt signal cascade, for enriching adult stem cells thus circumventing the need for serial transplantations in order to enrich stem cells. Thus, it can be concluded that certain signal cascades (for example, Wnt and hedgehog) are required for maintaining and expanding at least some stem cell populations.

The foregoing discussion demonstrates the need in the art for isolating tumor or metastatic cells from blood and analytical methods to investigate whether particular therapeutic agents are able to kill the tumor cells. To date, there are no simple clinical diagnostic methods to enable such a procedure to be carried out routinely with a high degree of sensitivity.

### SUMMARY OF THE INVENTION

The present inventors have addressed the foregoing need in the art by developing a novel approach for diagnosing tumor cells in a blood sample by transfecting the blood sample with nucleic acid constructs comprising reporter genes that are able to identify tumor cell-specific signalling activities in the blood sample. The method of the claimed invention has applicability for the detection of any disease-associated cell and is not exclusive to tumor cells. Further, the sample from which the cells are identified may include any body sample and is not exclusive to a blood sample.

The method of the present invention also has applicability in the isolation of stem cells from a heterogeneous cell mixture isolated from a living organism. In this context, the heterogeneous cell mixture is transfected with nucleic acid constructs comprising reporter genes that are detectable in stem cells. With this method, the stem cells are able to be isolated and used for clinical or therapeutic purposes.

In a preferred embodiment, the present invention provides a method for detecting disease-associated cells in a body sample comprising transfecting the body sample with at least one nucleic acid construct comprising: (a) a promoter element containing at least one DNA site for binding on or more transcription factors that initiates at least one signalling activity in the disease-associated cells that is not present or is greatly reduced in healthy cells; and (b) reporter genes, wherein expression of the reporter genes enables detection of the disease-associated cells.

In another embodiment, the present invention provides a method for isolating pluripotent stem cells from a body sample comprising transfecting cells isolated from the body sample with at least one nucleic acid construct comprising: (a) a promoter element containing at least one DNA site for binding one or more transcription factors that recognize signalling activities specific to stem cells; and (b) reporter genes, wherein expression of the reporter genes enables detection and isolation of the pluripotent stem cells. The functionality of the stem cells may be tested in vivo in immunosuppressed animal models and returned to the living organism from which the cells were isolated to regenerate identical organs or different organs after transdifferentiation.

### DETAILED DESCRIPTION OF THE INVENTION

### Definitions and Nomenclature

The term “reporter gene” encompasses coding nucleic acid segments that are introduced into cells and that do not naturally occur in the same form in the target cells that are to be analysed. These coding nucleic acid segments may include constituent segments or whole regions of naturally occurring sequences that, due to an altered sequence or a new context within the synthetic nucleic acid construct, produce products distinct from the natural gene products, or are expressed in an altered manner.

The term “reporter gene construct” encompasses nucleic acid constructs that minimally consist of a gene-regulating or recombinantly functional sequence and a coding sequence. The phrase refers particularly to DNA constructs comprising a gene-regulating sequence that directly (e.g., due to the presence of transcription factor-binding sites) or indirectly (e.g., by enabling the reporter gene to be specifically integrated, inserted, or recombined into genomic DNA segments) exerts an influence on the expression of the coding region. Gene-regulating sequences that directly influence the expression of a reporter gene are usually promoter, enhancer, or silencer regions, which through interactions with protein molecules are able to exert both positive and negative influences on the transcription of coding nucleic acid regions. Gene-regulating sequences that indirectly influence the expression of reporter genes include, as examples, (1) cases where a reporter gene is integrated into the genome of stem cell populations in which the target regions for the integrations have previously been specifically labelled by means of recombination steps (such as using loxP recombination sequences for the Cre recombinase) and the endogenous target genes may possibly, in addition to the recombination sequences, have been supplemented with one or more IRES sequences, and (2) cases where reporter genes contain IRES sequences such that additional reporter gene sequences are transcribed when expression of the endogenous gene locus is induced. If mammalian embryonic or partially differentiated stem cells are used for these purposes, the detection of specific cellular activities, including signalling activities, can be enabled using synthetic or endogenous gene-regulating sequences. Depending on the nature of the reporter gene, it is thus possible to detect the very early stages of pathological changes or of healthy physiological processes (such as differentiation, apoptotic, or proliferative processes). In this way, the effects of test substances can be analysed in model systems ranging from individual cells to entire recombinantly modified organisms; pathological changes can be observed at an early stage on the basis of reporter gene expression, and pathologically altered cells can be isolated if desired.

The term “signalling activity” encompasses biochemical activities in cells that ultimately regulate the expression of genes in the cell nucleus. Biochemical activities that lead to altered gene expression can take place at a very wide variety of intracellular sites, and are usually part of complex biochemical regulatory networks termed “signal transduction pathways.” In tumor cells, for example, receptors on the cell surface can be hyperactive due to increased ligand concentration, mutations in coding gene regions, or mutations in gene regulating regions. Proteins in the cytoplasm that participate in signal transduction pathways can exhibit altered activity due to mutations in the corresponding genes, such that post-translational modifications (e.g., phosphorylation, dephosphorylation, acetylation, deacetylation, or ubiquitinylation) are altered, as are resulting events (e.g., stabilization, destabilization, increased or decreased enzymatic activity, or decreased or augmented binding affinities). Ultimately, signalling activities occur through these pathways; the signalling activities increase or inhibit the expression of target genes.

The term “transcription factor” encompasses molecules, generally protein molecules, that associate directly or indirectly with nucleic acid regions and thereby elicit altered activities in these nucleic acid regions.

The term “expression level” refers to the amount of product resulting directly or indirectly from one or more coding nucleic acid sequences. This level may reflect transcriptional or translational activities that relate to nucleic acid regions that encode a sequence of amino acids or are complementary to a coding nucleic acid sequence. This level may also reflect post-translational modifications of amino acid sequences, which may result in their altered stability, location, or activity.

The term “cell-specific expression” encompasses the transcription and/or translation of coding sequence regions in particular cells in a heterogeneous cell population. For example, the cells may be pathologically altered cells that are present in a body sample containing healthy cells. The phrase particularly relates to the gene expressions in tumor cells that are due to the distinct biochemical activities in these cells. In this connection, reporter gene expression can achieve specificity by the cell-specific transcription, translation, or degradation of the reporter gene product or by the cell-specific transfection of the nucleic acid construct.

The term “body sample” encompasses any sample from an organism that contains living cells capable of being transfected with nucleic acid constructs. Examples of such body samples are blood, lymph fluid, stools, organ samples, and biopsies. Of particular interest are blood samples that are suspected to contain pathologically altered cells. Also of particular interest are biopsies, the cells of which are generally suitable for being transfected with nucleic acid constructs after they have been separately isolated using customary methods (e.g., cutting into pieces, resuspending with increasingly narrow needles, treating with protease).

The term “pathological change” encompasses abnormal biochemical activities in cells. Such abnormal activities usually are not concurrently present in comparable cells in the same tissue (healthy cells) and/or may be active in other regions of the tissue or in other phases of differentiation or stages of development.

The term “automatable method” encompasses methods in which the manual labour of human personnel, either entirely or only in constituent steps, is instead be performed by machines. Such methods may be included, as examples, in transfection, detection, isolation, documentation, and information processing.

### The Nucleic Acid Constructs:

By transfecting cells with synthetic nucleic acid constructs containing promoter elements and reporter genes, it is possible to use signalling activities in the cell nuclei of particular cells to specifically express the reporter genes. For example, following transfection of a heterogeneous cell mixture with synthetic nucleic acid constructs having promoter elements containing at least one transcription factor DNA binding site and reporter genes, reporter gene products are overexpressed or secreted in specific cells due to the concatameric juxtaposition of optimized transcription factor binding sites or the combination of different transcription factor binding sites. Thus, the use of the reporter genes makes it possible to use a variety of different detection methods.

The signalling activities in pathologically altered cells (e.g., tumor cells) may be used for specifically expressing fluorescent proteins. For example, since healthy cells in the blood sample do not possess signalling activities, the specificity of tumor cell detection is high and thus, living fluorescing tumor cells may be isolated by means of flow cytometry. The isolated tumor cells can be subsequently cultured and made available for further analyses. The nucleic acid constructs can also be used for cell culture systems that measure expression of the reporter genes before and after the transfected cells are contacted with potential active substances. This procedure can be used, on the one hand, for making individualized decisions with regard to therapy when the cells under investigation are derived from patients, and, on the other hand, assist in screening methods that analyze the activities of cells in particular cell lines.

Constituent regions of reporter genes encode biological activities that have a positive or negative influence on the initiation or progression of biological cascades. For example, as noted above, healthy cells in a blood sample do not possess the signalling activities that are required for expressing the reporter gene, or they possess these activities in significantly different degrees. Accordingly, in the method of the present invention, the biological cascades are only induced or inhibited in the presence of pathologically altered cells thereby leading to an extraordinarily high degree of specificity in tumor cell detection. The sensitivity of the tumor cell detection is very high because of the use of natural amplification systems or of biological cascades that are at least partially present in the blood. The tumor cell-specific expression of the reporter genes is subsequently detected by measuring the activation or of the biological cascades in question. In a blood sample, the reporter gene expression may be measured using classical blood coagulation assays (such as the Quick test or TPT). Pursuant to this method, then, tumor cells may be isolated from healthy cells in a blood sample.

Unlike reporter gene constructs used previously, which were used to study only one specific signal pathway, the nucleic acid constructs of the present invention may be used to analyze several signalling activities within a single cell. Further, when rare events, such as for example, metastasizing cells, are detected in blood, the possibility presents itself of using natural amplification systems that are present in blood. In particular, active components of the blood coagulation cascade may be made to be secreted specifically by metastatic tumor cells in patient blood samples, with these active components leading to the coagulation of tumor cell-containing blood samples. The diagnostic use of components of biological cascades as the reporter gene in blood samples is novel.

Within the context of the present invention, any one of the following components and substances may be used as reporter genes: active components of the blood coagulation cascade (such as thrombin, factor Va, factor VIIa, factor IXa, factor Xa, or factor XIIa); components of other biological cascades (such as the complement system of the kinin system); or substances that are generally active and that transform fibrin or other substrates (such as tPA, uPA, plasminogen/plasmin, or derivatives and/or hybrids thereof). Conversely, it is also possible to use variants of the components or substances that inhibit the biological cascades. Thus, for example, it would appear of value to use plasmin, i.e., the active component of plasminogen, as a secreted reporter gene since it inhibits blood coagulation (by altering the activity of factor X) while at the same time stimulating fibrinolysis. Consequently, plasmin is measurable under the corresponding experimental conditions of blood coagulation assays and transformation of chromogenic or fluorescent substrates. E. L. Pryzdial, N. Lavigne, N. Dupuis & G. E. Kessler, Plasmin Converts Factor X from Coagulation Zymogen to Fibrinolysis Cofactor, J. BIOL. CHEM. 274:8500-8505 (1999).

The method of the present invention as used for the detection of disease-associated cells in a body sample may be explained by way of the following. The contact of healthy body cells with pathogenic agents (such as infection with viruses, contact with bacteria or bacterial substances, or the presence or allergenic substances) or with stimulating or suppressing substances (such as growth factors, growth factor antagonists, toxins, and chemical substances) leads to changes in the biochemical activities of cell mixtures that facilitate the isolation of particular cells. Thus, it is conceivable, for example, that, while contact with growth-inhibiting substances suppresses particular biochemical activities in particular (e.g., healthy) cells, it has no effect in other cells (e.g., pathologically altered cells). The unsuppressed activities in these cells is then used for isolating the cells. The change in the uptake of particular substances may also be used for detecting specific cells in heterogeneous cell mixtures. Thus, as one example, it is possible to neutralize the increased expression of glutamate transporters in liver tumor cells for the purpose of isolating the cells. Where the transport proteins are expressed as reporter genes in target cells, increased transport of detectable (e.g., fluorescent) transport substrate analogues could be used for labelling the altered cells.

In addition to the foregoing, the method of the present invention may also be used to detect the presence of specific signalling activities in adult stem cells and thus to isolate the stem cells. For this purpose, nucleic acid constructs are used that are similar to those described for detecting and isolating tumor cells. Specifically, respective reporter genes are cloned downstream of promoter elements that are sensitive to the biochemical activities in adult stem cells. Preference is given to using nucleic acid constructs that encode fluorescent or transmembrane reporter gene products and that are introduced by means of viral expression systems into the cells in the body samples; however, it is also possible in principle to use other reporter genes. The cells are subsequently preferably isolated by flow cytometry in the presence of differentiation inhibitors, but other isolation methods may be used such as those that are based on detecting induced surface structures where the cells are isolated using beads. In addition to the foregoing, inhibitors of the differentiation processes (e.g., transcription factors having an appropriate effect on the expression of the genes of the respective stem cells) may also be expressed in a constitutive, induced, or cell type-specific manner in the stem cells, in addition to the reporter genes which are required for the cell isolation. The adult stem cells that have been isolated from a patient can, after surgical or other medical interventions, be used for forming or regenerating the identical organs or (after appropriate transdifferentiation) for regenerating other organs. For example, adult stem cells derived from the intestine could be used for regenerating the islet of Langerhans cells of the pancreas of patients suffering from diabetes.

Nucleic acid constructs are produced for expressing the reporter genes in body samples in a cell-specific manner, with the nucleic acid constructs containing the following components: (a) a promoter element that contains at least one, and preferably several, transcription factor DNA binding sites that are hyperactive in particular cells (due to, as examples, cell-specific activities or specific spatial arrangements on the DNA, such as concatamers), or else recombinantly relevant regions that influence the insertion or integration of nucleic acid constructs in target regions; and (b) a reporter gene that enables these specific cells to be detected. The cells are preferably transfected using viral expression systems, which enable cells present in the blood to be efficiently transfected with nucleic acid constructs. Particular preference is given to those transfection systems that, due to their cell-type preferences, bring about an additional specificity in the expression of the reporter gene. The level at which the reporter genes are expressed can be determined manually or through automatable methods.

Promoter elements can be designed to detect specific signalling activities. As a rule, transcription factor-binding sites for a specific signalling activity are spatially located as concatamers, which are separated by short DNA sequences (spacers) and are upstream from minimal promoters (e.g., from the endogenous c-Fos or thymidine kinase promoter). Several signalling activities can be measured simultaneously if DNA sites for binding transcription factors for different signal transduction pathways are combined with each other. Simultaneous and mutually independent measurements of different signalling activities in a cell can be achieved by transfecting nucleic acid constructs that each comprise a different reporter gene and different promoter elements. These transcription units, each consisting of a promoter region and a downstream reporter gene, can either be present on one DNA construct or on separate DNA constructs. It is commonly of particular value to use inducible (containing wild-type transcription factor-binding sites), non-inducible (containing mutated transcription factor-binding sites), and/or constitutively active nucleic acid constructs simultaneously, since this makes it possible to analyse the specific activation of a reporter gene by particular transcription factors or to determine transfection efficiencies and cytotoxicities. A single promoter can also simultaneously regulate the expression of two different reporter genes when the reporter genes are separated by an intervening IRES sequence (e.g., the internal ribosomal entry site sequence from the encephalomyocarditis virus).

It may sometimes be of value to supplement promoters with sites for binding basal transcription activators that fully display their transactivation potential only in the presence of specific disease-associated transcription factors. This cooperativity of transcription factors in activating promoters is based on findings from the MMTV promoter. For this promoter it was demonstrated that maximum stimulation following binding of the ligand-stimulated glucocorticoid receptor is only achieved by binding the basal transcription activator NF1. Activation of reporter gene expression can be augmented by establishing a positive feedback mechanism. For example, nucleic acid constructs can be made in which Wnt-sensitive promoters determine both the expression of a positive effector (for example, a fusion construct consisting of LEF-1 and the transactivating C-terminal region of β-catenin) and also the expression of the actual reporter gene, used for measuring Wnt signal transduction activity. K. Vleminckx, R. Kemler & A. Hecht, A., The C-Terminal Transactivation Domain of Beta-Catenin is Necessary and Sufficient for Signaling by the LEF-1/Beta-Catenin Complex in Xenopus Laevis, MECH. DEV. 81:65-74 (1999). In addition, the basal activity of synthetic promoter elements can be suppressed by adding DNA binding sites for transcription repressors. In this case, the corresponding transcription repressor can be present endogenously or be provided by adding a constitutively expressed repressor gene. It is also possible to use nucleic acid constructs that encode recombinant proteins that consist of a DNA-binding domain belonging to any desired transcription factor (e.g., the HMG domain belonging to LEF-1/TCF transcription factors, or the carboxy-terminal region (the last 90 amino acids) of c-myc) and a heterologous repressor domain (e.g., belonging to the Tet repressor, or the carboxy-terminal region (amino acids 179-281) of E2F6). The downstream reporter gene is only activated in the presence of a strong transcriptional activator on the same cis-acting promoter element, while the reporter gene is repressed in the absence of the transcription activator. Systems in which transcriptional repressors are regulated by endogenous activities (i.e., when a repressor gene is also used as a reporter gene in addition to the reporter genes that can actually be measured), are particularly interesting in this connection. Thus, for example, the expression of an exogenously introduced repressor gene can be regulated by the activity of the p53 transcription factor that is present in healthy cells. The repressor is only expressed in healthy cells and not in cells containing mutated p53; to test this it may be necessary to induce p53 activity by treating the cells appropriately (UV irradiation or the use of DNA-damaging substances). Accordingly, the expression of, for example, a Wnt-regulated reporter gene (e.g., GFP variants, luciferase, thromboplastin) that possesses transcription repressor-binding sites in the promoter region is not repressed in cells in which p53 is mutated, due to the absence of p53-regulated expression of the repressor gene; this means that the synergistic effect of Wnt signalling activity and p53 deficiency can be measured simultaneously or consecutively.

The p53 gene is a member of a transcription factor family that also includes the p63 and p73 genes. There are a variety of splicing variants of these genes, with these splicing variants differing, inter alia, in expression pattern, transactivation potential, repression potential, interactions with other proteins, and biochemical properties. A. Yang, M. Kaghad, Y. Wang, E. Gillett, M. D. Fleming, V. Dotsch, N. C. Andrews, D. Caput & F. McKeon, p63, a p53 Homolog at 3q27-29, Encodes Multiple Products with Transactivating, Death-Inducing, and Dominant-Negative Activities, MOL. CELL. BIOL. 2:305-316 (1998). Isoforms that lack the N-terminal transactivation domain are of importance. As dominant-negative agents, these proteins can exert a negative influence on the transactivation potential of the other isoforms on promoter elements by means of complex formation. The natural, and also the artificially produced, occurrence of these isoforms in mixed cell populations is of particular interest. Thus, it is known that, for example, N-terminally truncated p63 occurs specifically in certain basal cell populations, which may possibly contain stem cells or that are identical with stem cell populations. See, e.g., S. Signoretti, D. Waltregny, J. Dilks, B. Isaac, D. Lin, L. Garraway, A. Yang, R. Montironi, F. McKeon & M. Loda, p63 is a Prostate Basal Cell Marker and is Required for Prostate Development, AM. J. PATHOL. 157:1769-1775 (2000). Of particular interest is, for example, the reported occurrence of p63 in keratinocyte stem cells (G. Pellegrini, E. Dellambra, O. Golisano, E. Martinelli, L Fantozzi, S. Bondanza, D. Ponzin, F. McKeon & M. De Luca, p63 Identifies Keratinocyte Stem Cells, PROC. NATL. ACAD. SCI. USA. 98:3156-3161 (2001)). The occurrence of N-terminally truncated p63 in, for example, the stem cells of the skin can be used for isolating these cells, by inserting nucleic acid constructs with promoters that possess DNA sites for binding members of the p53 family into skin cell populations. As a result of the accumulation of induced p53 (from UV irradiation, nucleic acid-damaging agents, etc.), the reporter genes are expressed specifically in cells that express a dominant-interfering variant of one of the p53 family members, such as ΔN-p63 (or else, as in the case of abnormally altered tissue, exhibit a defect such as a p53 mutation). When a constitutively expressed reported gene is coexpressed, skin stem cells can, for example, be isolated in this way using the described methods.

The following transcription factors or families of transcription factors and/or responsive elements, inter alia, are examples of those of interest for measuring signalling activities in cells: TCF, LEF-1, jun, fos, myc, max, myb, E2F, DPI, CREB, p53, NFκB, NFAT, PPAR, ETS, ELK1, ATF, DPC, SMAD, CHOP, MEF, MADH4, GR, ER, STAT, SRF, ISRE, SRE, HSE, AP1, and CRE. Their transcription factor DNA binding sites have been described many times and are available to the public. Of particular interest in this connection are binding sites for the transcription factors TCF (5′-CCTTTGAA-3′ in J. J. Love, et al., Structural Basis for DNA Bending by the Architectural Transcription Factor LEF-1, NATURE 376: 791-795 (1995)); p53 (5′-PuPuPuC(A/T)(T/A)GpyPyPy-3′ in W. S. el-Deiry, S. E. Kern, J. A. Pietenpol, K. W. Kinzler & B. Vogelstein, B, Definition of a Consensus Binding Site for p53, NAT. GENET. 1:45-49 (1992)); PPARδ (5′-CGCTCAC-3; T. C. He et al., PPARdelta is an APC-Regulated Target of Nonsteroidal Anti-Inflammatory Drugs, supra); myc (5′-CACGTG-3′ or 5′-TCTCTTA-3′ in T. K. Blackwell, L. Kretzner, E. M. Blackwood, R. N. Eiseman & H. Weintraub, Sequence-Specific DNA Binding by the c-Myc Protein, SCIENCE 250:1149-1151 (1990)); E2F (5′-TTTTSSCGS-3′ or 5′-TTTCGCGC-3′ in M. Mudryj, S. W. Hiebert & J. R. Nevins, A Role for the Adenovirus Inducible E2F Transcription Factor in a Proliferation Dependent Signal Transduction Pathway, EMBO J. 9:2179-84 (1990)); AP1 (5′-TGA(C/G)TCA-3′ in T. M. Fisch, R. Prywes & R. G. Roeder, An AP1-Binding Site in the c-Fos Gene Can Mediate Induction by Epidermal Growth Factor and 12-O-Tetradecanoyl Phorbol-13-Acetate, MOL. CELL. BIOL. 9:1327-1331 (1989)); SMAD (5′-CAGACA-3′ in L. J. Jonk, S. Itoh, C. H. Heldin, P. ten Dijke & W. Kruijer, Identification and Functional Characterization of a Smad Binding Element (SBE) in the JunB Promoter that acts as a Transforming Growth Factor-Beta, Activin, and Bone Morphogenetic Protein-Inducible Enhancer, J. BIOL. CHEM. 273:21145-52 (1989)); and SRE and ATF (T. M. Fisch, R. Prywes, M. C. Simon & R. G. Roeder, Multiple Sequence Elements in the c-Fos Promoter Mediate Induction by cAMP, GENES DEV. 3:198-211 (1989)).

In order to detect and/or isolate fluorescent cells, it is necessary to use reporter genes that encode fluorescent proteins. In principle, all genes that directly or indirectly encode fluorescent proteins are suitable for this purpose. Variants of the following genes are particularly suitable: GFP, BFP, YFP, CFP, DS-Red, obilin, and aequorin. In addition to this, it is also possible to use genes that encode fluorescent dye-binding proteins. As one example, a gene may encode anticalins, which bind the fluorescent dye FITC. Alternatively, however, it is also possible to use coding nucleic acid segments whose translated products are exposed on the cell surface and are consequently available for secondary detection methods. By this is meant, in particular, those nucleic acid segments that encode transmembrane proteins or molecules that are presented extracellularly, or which lead to these molecules being exposed. For example, antigens, receptors, or ligands for which specific detection molecules (e.g., antibodies, anticalins, ligands, or receptors) are available can be presented on the cell surface as a result of the signalling activities. In this connection, for diagnostic methods preference is given to those coding nucleic acid segments that are not of human origin. For example, in immunological detection methods it is possible to use homologous gene segments encoding transmembrane proteins, which are also found in humans, from the mouse, rat, etc. in order to suppress cross reactivities, which are due to endogenous expression in nontransfected cells. It is also possible, however, to use synthetic sequences for which corresponding detection molecules are prepared. For example, it is possible to use molecular biological methods to recombine virtually any synthetic peptide sequences with transmembrane proteins; such sequences are presented on the cell surface in isolated or concatameric form. At the same time, it is possible to generate highly specific antibodies directed against these peptides. By coupling these detection molecules to different magnetic or non-magnetic beads, it is possible to isolate the pathologically altered cells on the basis of the specific expression of the exogenous nucleic acid region, by applying magnetic fields or by centrifugation steps, due to the pathological cells binding to the beads.

In order to detect specific signalling activities associated with biological cascades, it is necessary to use reporter genes that are equivalent to activating or inhibitory components of the biological cascades. In principle, it is possible to use, as examples, sequence regions of the genes prothrombin/thrombin, factor XIIa, factor XIa, factor Xa, factor IXa, factor VIIIa, factor VIIa, factor Va, fibrin/fibrinogen, plasmin/plasminogen, prokallikrein/kallikrein, urokinase, tPA, CVF, C3b, protein C, C-1 S inhibitor, hirudin, α-1-antitrypsin, AT-III, TFPI, PAI-1, PAI-2, or PAI-3 for this purpose. In addition to this, it is also possible for enzymatic proteins to be expressed or corresponding fusion proteins to be activated by the biological cascades.

For the cell-specific expression of reporter genes, the DNA constructs must be introduced into the cells. A large number of commercially available transfection technologies have been developed for this purpose. In addition to classical calcium phosphate precipitation, direct transfer by means of microprojectiles (also called the shot-gun approach), and electroporation methods, it is possible to transfect mammalian cells using liposome technologies (e.g., lipofectin and lipofectamine from GibcoBRL). Mammalian cells, however, can be particularly efficiently transfected using viral systems. In particular, systems have been established that are based on retroviral, adenoviral, or adeno-associated viral (“AAV”) vectors. An interesting approach in this connection is to use transfection systems that achieve different transfection efficiencies in different cell types, and consequently promote specificity of the reporter gene expression. For example, adenoviruses infect with extremely high efficiency most human cells, except hematopoietic cells. By selecting appropriate incubation conditions, it is possible to exploit this fact when isolating epithelial cells from the blood, thereby enabling tumor cells to be enriched even when using constitutive reporter gene expressions.

Thus, within the context of the method of the present invention as used for the detection of the disease-associated cells, the nucleic acid constructs are comprised of the following: (a) promoter elements that possess sites for binding transcription factors that are at least partly responsible for altered gene expressions in diseased cells or for gene expression that is different from that in healthy cells; or (b) reporter genes that produce products readily measurable and not normally not present in the other body cells in the same form or quantity. The specificity and sensitivity of the inducible reporter gene activation is ensured by the combination and number of transcription factor-binding sites. By means of expressing a fluorescent protein that is not endogenously present in human cells, the method, which is specific and which can be automated by means of flow cytometry, can be performed qualitatively or quantitatively. In addition, by specifying threshold values for fluorescent intensities, flow cytometry makes it possible to isolate cells that exhibit particular levels of reporter gene expression. Furthermore, the simultaneous measurement of two different fluorescent proteins, which can each be induced by different biochemical activities, enables individual tumor cells to be characterized more precisely with regard to their degree of degeneration (i.e., staging). This applies, in particular, to those tumor types in which the corresponding biochemical activities occur in different stages of the tumor progression (as explained in the Background section using the example of colon carcinoma). The simultaneous use of two similar, but not identical, reporter genes can also serve to determine the specificity of reporter gene expression. Thus, promoters that contain intact or mutated transcription factor-binding sites can, for example, regulate the expression of reporter genes that encode proteins whose fluorescence differs or of enzymes whose substrate specificity differs. As a result, comparison of expression levels enables conclusions to be drawn with regard to the activity of specific signalling activities. It also may be of value to use, in addition to inducible promoters, constitutively active promoters that regulate different reporter genes, in order to monitor transfection efficiencies or cytotoxicities.

Utility:

The present invention has utility as a method for diagnosing disease-associated cells. By transfecting nucleic acid constructs that contain inducible promoter elements into mixtures of tumor cells and healthy cells, tumor cells may be detected and isolated from the healthy cells in an automatable manner. The invention also has utility in enabling specific, healthy cells (e.g., stem cells) to be isolated from heterogeneous cell mixtures or body samples.

The use of signalling activities in mammalian cells to express reporter genes, which enables particular cells to be detected and isolated, is advantageous both for diagnostic and therapeutic purposes. Uses for which this technology can be applied to include the detection of diseased cells in body samples, the isolation of such cells from these samples, monitoring changes during therapy, drug screening, and the testing of patient samples ex vivo for therapeutic purposes. Other areas of application include stem cell technology, in which stem cells can be isolated, and, where appropriate, differentiated or transdifferentiated, and used for growing replacement organs or else for regenerative processes.

It is to be understood that while the invention has been described in conjunction with the preferred specific embodiments thereof, that the description above as well as the examples which follow are intended to illustrate and not limit the scope of the invention. Other aspects, advantages and modifications within the scope of the invention will be apparent to those skilled in the art to which the invention pertains.

All patents, patent applications, and publications mentioned herein, both supra and infra, are hereby incorporated by reference in their entireties.

### EXPERIMENTAL

All the molecular biological standard methods that are mentioned in the Examples, but that are not described in detail (such as plasmid DNA preparations on an analytical scale, the cleavage of DNA with restriction endonucleases, the dephosphorylation of linearized DNA, the filling-in of protruding ends, the ligation of the DNA molecules, the transformation of bacteria, the fractionation of nucleic acids on agarose gels, etc.) are well known in the art and are described in the literature, such as: J. Sambrook, E. F. Fritsch, and T. Maniatis, Molecular Cloning: A Laboratory Manual (Cold Spring Harbor Laboratory Press, Cold Spring Harbor, N.Y., 2nd ed. 1989).

### Example 1

### Overexpression of αGFPT204I in SW480 Colon Carcinoma Cells on the Basis of Tumor-Associated Wnt Signal Activity in these Cells

In order to express a variant of the GFP αGFPT204I in a tumor-specific manner, Wnt-responsive reporter gene constructs are first prepared. The promoter regions of these constructs contain three functional or three non-functional sites for binding LEF-1/TCF transcription factors (CCTTTGATC and CCTTTGGCC, respectively), or variants of these binding sites (CCTTTGAA or CCATTGAA and CCTTTGGA or CCATTGGA, respectively). In order to generate Wnt-responsive GFP reporter gene constructs, the literature-describes starting vectors pTOPFLASH, pFOPFLASH, pTOPCAT, and pFOPCAT are first used (see M. Van De Wetering, R. Cavallo, D. Dooijes, M. Van Beest, J. Van Es, J. Loureiro, A. Ypma, D. Hursch, T. Jones, A. Brjsovec, M. Peifer, M. Mortin, and H. Clevers, Armadillo Coactivates Transcription Driven by the Product of the Drosophila Segment Polarity Gene dTCF, CELL 88:789-799 (1997); and M. Van De Wetering, M. Oosterwegel, D. Dooijes, and H. Clevers, Identification and Cloning of TCF-1, A T Lymphocyte Specific Transcription Factor Containing a Sequence-Specific HMG Box, EMBO J. 10:123-132 (1991)). In subsequent experiments, the promoter region is augmented by ligating specially synthesized, double-stranded oligonucleotides containing the binding sites for LEF-1, PPARδ, myc, etc.

In the case of the pTOPFLASH and pFOPFLASH vectors, the luciferase gene is excised by digesting with the restriction enzymes NCO1 and NOT1 and the vectors are subsequently dephosphorylated by incubating them with alkaline phosphatase. The reaction products are then separated by gel electrophoresis in ethidium bromide-containing agarose gels, after which the vector band, of approximately 3.8 kb in size, is excised from the gel and the DNA is isolated using the “Qiaex® II gel extraction kit” supplied by Qiagen. The αGFPT204I gene is amplified by means of the PCR reaction and restriction cleavage sites are introduced from the pKU23 vector or the α+GFPcycle3 vector supplied by Maxygen, which contains the αGFPT204I gene; the restriction sites are as follows:

NCO1 for the 5′-located primer
5′-CCC GGG CC ATG GCT AGC AAA GGA GAA GAA CTT TTC
AC-3′;
and
NOT1 for the 3′-located primer
5′-GGC CGC GGC CGC TTA TTT GTA GAG CTC ATC CAT
GCC-3′).

See, A. Crameri et al., Improved Green Fluorescent Protein By Molecular Evolution Using DNA Shuffling, supra. The resulting products are then digested with the restriction endonucleases NCO1 and NOT1, purified by treatment with phenol/chloroform, precipitated (addition of 1/10th vol of 3M NaAc and 2.5 vol of 100% ethanol), washed with 70% ethanol, dried and taken up in 1× tris/EDTA buffer. The reaction product, of approximately 700 bp in size, is subsequently separated by gel electrophoresis and isolated from the agarose gel. The TOP and/or FOP vector(s) is/are ligated to the amplified αGFPT204I cDNA using T4-DNA ligase in an appropriate buffer containing Mg²⁺ and ATP. The ligation mixtures are transformed into bacteria (e.g., using DH5α™ Competent Cells from GibcoBRL Life Technologies), with these bacteria subsequently being streaked out on ampicillin-containing LB agar plates. Ampicillin-resistant single colonies are used for inoculating 5 mL volumes of LB liquid cultures, after which the plasmid DNA is isolated from grown single colonies (e.g., using Concert™ Rapid Plasmid Mini Prep System from GibcoBRL Life Technologies), analysed by restriction digestion with NOT1 and NCO1, and sequenced (e.g., using Big Dye™ Terminator Cycle Sequencing Ready Reaction from PE Applied Biosystems) using several sense and antisense primers, and verified. The plasmid DNA having the desired sequence is subsequently produced in larger quantities and purified (e.g., using QIAfilter Plasmid Maxi Kit from Qiagen), and then used for transfecting SW480 colon carcinoma cells.

The reporter gene constructs are transfected, as desired, using Lipofectin® reagent, Lipofectamine Plus™ reagent, or Lipofectamine™ 2000 reagent in accordance with the instructions from the manufacturing company, GibcoBRL Life Technologies, with the SW480 cells being seeded prior to the transfection in 6-well plates (approximately 300,000 cells per well) and incubated overnight in DMEM, 10% fetal calf serum, 100 μg of penicillin at 37° C., 90% atmospheric humidity and a content of 7.5% CO₂. Between 1 μg and 10 μg of the TOP-αGFPT204I vector DNA, the FOP-αGFPT204I vector DNA, and for the control, the α+GFPcycle3 vector DNA, are mixed thoroughly with 10 μl of Lipofectin or Lipofectamine and 90 μl of Optimem in polystyrene tubes and the mixtures are incubated at room temperature for 1 hour. Subsequently, 800 μl of Optimem is added and, following a one-hour incubation, the mixtures are added to the cells, which have been washed several times with PBS; the cells are then incubated overnight. On the following morning, the transfected SW480 cells are washed once again with PBS and incubated again in DMEM+10% FCS. Following appropriate excitation under a fluorescence microscope (e.g., using Olympus AX70 having a Multicontrol Box 200-3 and Module Stage analySIS driver software), the cells that have been transfected with α+GFPcycle3 and TOP-αGFPT204I begin to emit light whereas the cells transfected with FOP-αGFPT204I do not exhibit any significantly increased fluorescence. These findings are confirmed by measuring the fluorescence intensities in a flow cytometer (e.g., using FACSscan, Becton Dickinson), which is equipped with laser excitation for green fluorescence. The fluorescence intensities are plotted, with defined threshold values for fluorescence intensities being assigned to the positive signals. In order to optimize the GFP fluorescence detection, the standard fluorescein filter is, where appropriate, replaced with broad band violet filters (e.g., using U-MBV from Olympus), which enable excitation to take place between 340 and 440 nm and detection to take place from 475 and 490 nm, respectively.

### Example 2

### Overexpression of αGFPT204I in SW480 Cells on the Basis of the Combinatorial Use of Transcription Factor-Binding Sites for LEF-1/TCF and PPARδ

Because of Wnt signal activity, SW480 cells overexpress the transcription factor PPARδ (T. C. He, T. A. Chan, B. Vogelstein & K. W. Kinzler K., PPARdelta is an APC-Regulated Target of Nonsteroidal Anti-Inflammatory Drugs, CELL 99:335-345 (1999)). In order to measure the synergistic effect of transcription factor-binding sites when expressing reporter genes, PPARδ-responsive elements are cloned into the TOP-αGFPT204I vector and the FOP-αGFPT204I vector. For this, the following oligonucleotides are dimerized:

functional PPARδ DNA binding sites
5′-CTAGCGTGAGCGCTCACAGGTCAATTCGGTGAGCGCTCACAGGTCAA
TTCG-3′;
or
functional PPARδ and PPARδ DNA binding sites
5′-CTAGCGGACCAGGACAAAGGTCACGTTCGGACCAGGACAAAGGTCAC
GTTCG-3′

after thoroughly mixing with equal quantities of correspondingly synthesized, complementary counterstrand oligonucleotides, by heating and cooling, and inserted upstream, 5′ of the functional and/or nonfunctional LEF-1/TCF-binding sites, into the promoter region of the reporter gene construct by means of blunt-end ligation. The ligation mixtures are transformed into bacteria, which are then streaked out on ampicillin-containing LB agar plates; the plasmid DNA is isolated from grown single colonies, analysed by restriction digestion with NOT1 and NCO1 and sequencing using several sense and antisense primers, and verified. The plasmid DNA having the desired sequence is subsequently produced in larger quantities and purified, and used for transfecting SW480 colon carcinoma cells, employing lipofectin or lipofectamine. Measurement of the fluorescent intensities in a flow cytometer indicates a measurable synergistic effect with regard to an increased expression, or fluorescence intensity, of αGFPT204I in the presence of functional LEF-1/TCF- and PPARδ DNA binding sites. In addition to this, it is found that a 10 hour incubation of transfected cells with NSAIDs (i.e., 300 μM Sulindac sulphide, indomethacin, or aspirin) significantly inhibits the observed activation of the FOP-αGFPT204I reporter gene in the presence of functional PPARδ-DNA binding sites such that the expression of αGFPT204I returns virtually to the background level of the FOP-αGFPT204I reporter gene activity without any functional PPARδ DNA binding sites.

### Example 3

### Isolating Colon Carcinoma Cells from Heterogeneous Cell Mixtures by Means of Flow Cytometry

In order to isolate fluorescent SW480 cells that have been transfected in the above-described manner with TOP-αGFPT204I or α+GFPcycle3, SW480 cells are released from the substratum by adding trypsin solution (0.5 mM EDTA and 2% trypsin in PBS) and resuspended in DMEM+10% FCS. The cells are subsequently taken up, as desired, in ISOTON II (Coulter) or measured directly in a flow cytometer (FACSscan, Becton Dickinson). Cells that fluoresce above a defined signal intensity are isolated and mixed, in a defined numerical portion, with cell suspensions of transfected or untransfected cells derived from another type of tissue (i.e., not colon; e.g., IIA1.6 B cells, C57MG breast tumor cells, Jurkat cells, or BW5147 T cells), whose cells do not possess any Wnt signal activity and consequently do not exhibit any significantly increased fluorescence in experiments (as explained in Example 1) following transfection with TOP-αGFPT204I as compared with fluorescence following transfection with FOP-αGFPT204I. These heterogeneous cell mixtures are subsequently measured once again in the flow cytometer and the number of detected fluorescent cells is compared with the number of fluorescent SW480 cells that were originally added (equal to the recovery rate).

### Example 4

### Isolating Colon Carcinoma Cells from Heterogeneous Cell Mixtures by Means of Flow Cytometry after Transfecting Heterogeneous Cell Populations

HeLa cervix carcinoma cells, 3T3 fibroblasts, or SV40-transformed COS cells are mixed, in different quantitative proportions, with SW480 colon carcinoma cells or left as a homogeneous cell population for control purposes. Next, cells of the heterogeneous cell mixture, or of the homogeneous cell populations, are seeded at 300,000 cells per well in the wells of 6-well plates and incubated overnight in DMEM, 10% fetal calf serum, 100 μg of penicillin at 37° C., 90% atmospheric humidity and a CO₂ content of 7.5%. The plasmids TOP-αGFPT204I, FOP-αGFPT204I, and α+GFPcycle3 are subsequently transfected, as described in Example 1, using the lipofectin and lipofectamine reagents. At different times after the transfection (4, 16, 24, and 48 hours), the cells are released by treating with trypsin and measured by means of flow cytometry. The number of detected fluorescent cells per mixture is compared with the number of SW480 cells originally added in order to determined the specificity and sensitivity of the tumor cell detection.

### Example 5

### Adenoviral Transfection of Heterogeneous Cell Populations Consisting of Colon Carcinoma Cells Possessing Wnt Signal Activity and Control Cells Lacking Wnt Signal Activity, and Subsequent Flow Cytometry

In order to increase transfection efficiencies, adenoviral reporter gene constructs are prepared using the “Adeno-X TM Expression System” supplied by Clontech (Cat. No. K1650-1). For this purpose, the promoter region, including the αGFPT204I sequence, is amplified by the PCR reaction from the TOP-αGFPT204I and FOP-αGFPT204I vectors. In this connection, use is made of primers that introduce restriction cleavage sites for the enzymes I-CEU I and NOT1 at the 5′ and 3′ ends, respectively, of the reaction product, such as for example:

5′sense primer
5′-CCC GGG TAA CTA TAA CGG TCC TAA GGT AGC GAG CAA
TTG TTG TTA ACT TGT TTA TTG CAG CTT ATA ATG G-3′;
and
3′ antisense primer
5′-GGC CGC GGC CGC TTA TTT GTA GAG CTC ATC CAT
GGC-3′.

The reaction products are subsequently digested with the restriction antinucleases I-CEU I and NOT1, purified by treatment with phenol/chloroform, precipitated (addition of 1/10th vol of 3M NaAc and 2.5 vol of 100% ethanol), washed with 70% ethanol, dried and taken up in 1× tris/EDTA buffer. Following gel-electrophoretic purification, the reaction product is isolated from the agarose gel. The pShuttle vector, which is provided by Clontech, is correspondingly digested with the enzymes I-CEU I and NOT1, dephosphorylated, separated gel-electrophoretically, and extracted from the gel (as described in Example 1). The pShuttle vector is ligated to the amplified TOP-αGFPT204I sequence or FOP-αGFPT204I sequence using T4-DNA ligase in an appropriate buffer containing Mg²⁺ and ATP. The ligation mixtures are subsequently transformed into bacteria, which are then streaked out on ampicillin-containing LB agar plates; the plasmid DNA is isolated from grown single colonies, analysed by restriction digestion and sequencing using several sense and antisense primers, and verified. The plasmid DNA of the desired sequence is produced in relatively large quantities and purified.

The subsequent steps take place in accordance with the instructions provided by the manufacturer Clontech (see, Adeno-X TM Expression System User Manual). The TOP-αGFPT204I sequence or the FOP-αGFPT204I sequence is excised from the pShuttle vector using the restriction enzymes I-CEUI and PI-SCEI and ligated to the correspondingly digested adeno-X virus DNA (in accordance with the customary molecular biological intermediate steps, as described above). The in vitro ligation is subsequently digested with the restriction enzyme SWAI and the corresponding mixture is used for transforming bacteria. DNA preparations from ampicillin-resistant transformants are analysed by means of suitable restriction digestion and sequencing using several sense and antisense primers, and verified. The recombinant adenoviral plasmid DNA of the desired sequence is subsequently prepared on a small scale in accordance with the instructions in the Clontech manual and produced on a large scale using the NucleoBond® Plasmid Maxi Kit in accordance with the Clontech instructions, and purified. The purified plasmid DNA is digested with the restriction enzyme PACI and, following purification of the reaction mixture, used for transfecting HEK 293 cells employing lipofectamine or lipofectin. After from 4 to 7 days, a large number of the cells become detached such that the supernatant, which contains recombinant adenoviruses possessing the TOP-αGFPT204I sequence or the FOP-αGFPT204I sequence, can be used, after centrifugation, for infecting the target cells.

In accordance with Examples 2 and 3, homogeneous cell populations of SW480 cells are then incubated with the recombinant adenoviruses. For this purpose, approximately 5×10⁵ SW480 cells are seeded in 100 mm cell culture dishes so as to enable them to adhere to the bottoms of the cell culture dishes overnight (or else for up to 48 hours). Subsequently, 1 mL of the virus-containing medium is added to the cells for approximately 1 hour. After that, the cells are washed once with DMEM+10% FCS and incubated for up to 48 hours in an incubator. After 2, 4, 8, 16, 24, and 48 hours, the expression of GFP is measured by means of fluorescence microscopy and flow cytometry. The number of GFP-expressing cells following infection with the TOP-αGFPT204I sequence is extremely high (70-95% after 24 hours), whereas cells transfected with the FOP-αGFPT204I sequence do not exhibit any significant GFP expression. The infection of heterogeneous cell mixtures leads to specific overexpression of the GFP gene, following infection with the TOP-αGFPT204I sequence, in the colon carcinoma cells, which possess an active Wnt signal activity. In the next step, SW480 cells are added, in different quantities, to 1 mL of human whole blood samples from healthy persons, and 1 mL of virus-containing medium is then added to the cell suspension for 1 hour. The samples are washed by several careful centrifugation steps and a change of medium and are incubated for a further 12 to 18 hours at 37° C. with gentle agitation in an incubator. The expression of GFP is subsequently measured by means of flow cytometry and the number of positive fluorescent signals is correlated with the number of SW480 cells that have in each case been added. In addition to this, the fluorescent cells are isolated using appropriate threshold value settings and identified as epithelial (i.e., colon carcinoma) cells purely by morphological inspection or by means of a standard immunocytochemical method, provided by Miltenyi Biotec (Cat. No. 603-01) using highly specific cytokeratin-FITC antibodies and anti-Fitc alkaline phosphatase in accordance with the manufacturer's instructions.

### Example 6

### Overexpression of STF-LZ in SW480 Colon Carcinoma Cells on the Basis of the Tumor-Associated Wnt Signal Activity in these Cells

In order to express in a tumor-specific manner a fusion product consisting of soluble thromboplastin (“soluble tissue factor” or “STF”; amino acids 1 to 220) and the leucine zipper domain (“leucine zipper” or “LZ”) from the yeast transcription factor GCN4, Wnt-responsive reporter gene constructs are first prepared. The promoter regions contain three functional or three non-functional sites for binding LEF-1/TCF transcription factors (CCTTTGATC and CCTTTGGCC, respectively), or variants of these binding sites (CCTTTGAA or CCATTGAA and CCTTTGGA or CCATTGGA, respectively). In order to generate Wnt-responsive sTF-LZ reporter gene constructs, the literature-described starting vectors pTOPFLASH, pFOPFLASH, pTOPCAT and pFOPCAT are used. M. van de Wetering, R. Cavallo, D. Dooijes, M. Van Beest, J. Van Es, J. Loureiro, A. Ypma, D. Hursch, T. Jones, A. Brjsovec, M. Peifer, M. Mortin & H. Clevers, Armadillo Coactivates Transcription Driven by the Product of the Drosophila Segment Polarity Gene Dtcf, CELL 88:789-799 (1997); M. van de Wetering, M. Oosterwegel, D. Dooijes & H. Clevers, Identification and Cloning of TCF-1, a T Lymphocyte Specific Transcription Factor Containing a Sequence-Specific HMG Box. EMBO J. 10:123-132 (1991). In the case of the pTOP-FLASH and pFOP-FLASH vectors, the luciferase gene is excised by digesting with the restriction enzymes NCO1 and NOT1 and the vectors are subsequently dephosphorylated by incubating them with alkaline phosphatase. After that, the reaction products are separated gel-electrophoretically in ethidium bromide-containing agarose gels, after which the vector band, of approximately 3.8 kb in size, is excised from the gel and the DNA is isolated using the Qiaex® II Gel Extraction Kit supplied by Qiagen.

The cDNA for the leucine zipper domain is amplified by the polymerase chain reaction (PCR) from genomic yeast DNA using the following primer pair:

coding sense primer
5′-ATC GGC GGC GCC GCC ATG AAA CAA CTT GAA GAC
AAG-3′;
and
antisense primer
5′-GAT CAA AGC TTG CGG CCG CTC AGC GTT CGC CAA CTA
A-3′.

In this connection, the coding sense primer contains an Nar1 restriction enzyme cleavage site and, in addition to this, encodes a short linker sequence, i.e., Gly-Gly-Ala-Ala, which is located upstream of the leucine zipper sequence Met-Lys-Asn-Leu. For subsequent cloning steps, the antisense primer contains cleaving sites for the restriction enzymes Hind3 and NOT1. The cDNA for soluble thromboplastin (amino acids 1-220 with and without signal sequence) is amplified by PCR using the following coding sequence primers:

5′-GAA GAA GGG ATC CTG GTG CCT CGT GGT TCT GCC ATG
GGC ACT ACA AAT ACT GTG GCA GC-3′;
and
5′-GAA GAA GGG ATC CTG GTG CCT CGT GGT TCT CC ATG
GAG ACC CCT GCC TGG CCC CGG G-3′,

and the antisense primers:

5′-GGC GGC GCC GCC TAT TTC TCG AAT TCC CC-3′;
(codon 226 -> F)
5′-GGC GGC GCC GCC TAT TTC TCG CCC ATA CAC TCT ACC
GGG CTG TCT G-3′; (codon 220 -> G)
and
5′-GGC GGC GCC GCC TAT TTC TCC TCT ACC GGG CTG TCT
GTA CTC TTC CGG-3′. (codon 217 -> E)

In this connection, the sense primers contain, for subsequent cloning steps, the cleavage sites for the restriction enzymes BamH1 and NOT1. The antisense primers contain an Nar1 cleavage site for the fusion with the GCN4 leucine zipper domain. After having been digested with the corresponding restriction enzymes, purified by treatment with phenol/chloroform, precipitated (i.e., addition of 1/10th vol of 3M NaAc and 2.5 vol of 100% ethanol) and washed with 70% ethanol, the PCR fragments are dried and taken up with 1× tris/EDTA buffer.

The reaction products are subsequently separated gel-electrophoretically and isolated from the agarose gels. The ligation of the PCR products with the BantH1-cut, Hind3-cut, dephosphorylated, gel-electrophoretically separated, and subsequently isolated pTrcHisC vector is effected using T4 DNA ligase in an appropriate buffer containing Mg²⁺ and ATP. See, M. J. Stone, M. Ruf, D. J. Miles, T. S. Edgington & P. E. Wright, BIOCHEM. J. 310:605-614 (1995). The ligation mixtures are transformed into bacteria (e.g., using DH5α™ competent cells from GibcoBRL Life Technologies), which are streaked out on ampicillin-containing LB agar plates. Grown, ampicillin-resistant single colonies are used for inoculating 5 mL LB liquid cultures, and the plasmid DNA is isolated from the grown single colonies (Concert™ Rapid Plasmid Mini Prep System from GibcoBRL Life Technologies) and is analysed and verified by means of restriction digestion and sequencing (Big Dye™ Terminator Cycle Sequencing Ready Reaction from PE Applied Biosystems) using several sense and antisense primers. The plasmid DNA having the desired sequence is subsequently produced in larger quantities, purified (e.g., using QIAfilter Plasmid Maxi Kit from Qiagen), and used for expressing the fusion protein in Escherichia coli.

Bacterially expressed protein is extracted from the bacteria using guanidium hydrochloride (“GuHCl”) and, after having been purified through Ni-chelate columns (e.g., using Ni-NTA from Qiagen), is folded on the column using a linear gradient of buffer A (6M GuHCl; 0.5M NaCl; 20 mM sodium phosphate; pH 8) and buffer B (0.8M GuHCl; 0.3M NaCl; 50 mM tris-HCl; 2.5 mM reducing glutathione; 0.5M oxidizing glutathione; pH 8). After intensive washing with 10 mM Tris/20 mM NaCl/pH 7.5, the protein is eluted in the same buffer, which additionally contains 50 mM imidazole, and is used for demonstrating the functionality of the fusion protein or as a positive control in blood coagulation assays.

By means of digesting with the restriction endonucleases NCO1 and NOT1, the fusion gene is excised from the pTrcHisC-sTF-LZ expression vector and ligated into the correspondingly digested TOP/FOP vectors. After the vectors have been transformed into bacteria, single colonies that have grown on agar plates are replicated in the above-described manner and analysed. Bacterial clones that harbour the desired nucleic acid constructs are produced in larger quantities and the respective plasmid DNA is purified and used for transfecting SW480 cells.

The reporter gene constructs are transfected, as desired, using Lipofectin® reagent, Lipofectamine Plus™ reagent, or Lipofectamine™ 2000 reagent in accordance with the instructions of the manufacturer, GibcoBRL Life Technologies, with the SW480 cells being seeded, prior to the transfection, in 6-well plates (having approximately 300,000 cells per well) and incubated overnight in DMEM, 10% fetal calf serum, 100 μg of penicillin at 37° C., 90% atmospheric humidity and a CO₂ content of 7.5%. Between 1 μg and 10 μg of the TOP-sTF-Lz and FOP-sTF-LZ vector DNAs are thoroughly mixed with 10 μL of lipofectin or lipofectamine and 90 μL of Optimem in small polystyrene tubes and incubated at room temperature for 1 hour; 800 μL of Optimem are subsequently added and, after a one-hour incubation, the mixtures are added to the cells, which have been washed several times with PBS, and the cells are incubated overnight. The following morning, the transfected SW480 cells are washed once again with PBS and incubated again in DMEM+10% FCS. The cell culture supernatants from parallel batches of the SW480 cells transfected with FOP-sTF-LZ/TOP-sTF-LZ are removed at different times (12, 24, and 36 hours after transfection) and freed of cell residues by being centrifuged at 10,000×g. The supernatants are subsequently, in initial experiments, adjusted to trisHCl (pH 7.4) and EDTA concentrations, by adding these compounds, of 50 mM and 10 mM respectively, and concentrated ten times by filter centrifugation (e.g., usng S1Y10 Spiral Ultracentrifugation Cartridge from Amicon). These concentrated cell culture supernatants are either stored at −80° C. or used directly for blood coagulation assays. Supernatants that contain sTF₂₁₇-LZ, sTF₂₂₀-LZ or sTF₂₂₆-LZ are added to aliquots of pooled human blood samples (citrate whole blood), some of which samples additionally contain factor VIIa and 28 μM of a phosphatidyl serine/phosphatidyl choline mixture (40% PS to 60% PC in TBS containing 0.1% BSA). The time periods that elapse until the different blood sample mixtures coagulate following recalcification are measured manually. It is found that the addition of cell culture supernatants from the SW480 cells transfected with TOP-sTF-LZ leads to a significantly accelerated coagulation of the blood sample in question, with sTF₂₁₂-LZ and sTF₂₂₀-LZ being particularly active. By contrast, the blood coagulation times following the addition of cell culture supernatants from the SW480 cells transfected with FOP-sTF-LZ are significantly altered as compared with control mixtures to which no cell culture supernatants are added.

### Example 7

### Detecting TOP-sTF-LZ Transfected Cells in Blood Samples

SW480 cells are first cotransfected, in the manner described in Example 1, with the plasmids a+GFPcycle3 and TOP-sTF-LZ or FOP-sTF-LZ. After that, the SW480 cells are released from the substratum by adding trypsin solution (0.5 mM EDTA and 2% trypsin in PBS) and resuspended in DMEM+10% FCS. Cells that fluoresce above a defined signal intensity are isolated by flow cytometry and added, in a defined numerical proportion, to aliquots of pooled human blood samples. The periods of time that elapse until the different blood sample mixtures have coagulated following recalcification are measured manually. It is found that the decrease in the blood coagulation times is directly related to the quantity of TOP-sTF-LZ-transfected SW480 cells added. On the other hand, the addition of FOP-sTF-LZ-transfected SW480 cells does not exhibit nearly as great a reduction in the blood coagulation times.

1.-42. (canceled)

43. A method of measuring a level of promoter activity in a cell comprising:
transfecting a cell from a sample of human cells with a synthetic nucleic acid construct comprising
a promoter element and
a reporter gene, wherein said promoter element has at least one transcription factor binding site and said reporter gene encodes a fluorescent protein and measuring the level of the reporter gene products in the cell to detect the promoter activity in the cell.
